(19) United States
(12) Patent Application Publication
(10) Pub. No.: US 2014/0066687 A1
(43) Pub. Date: Mar. 6, 2014

(54) RADIATION THERAPY OF PROTRUDING AND/OR CONFORMABLE ORGANS

(71) Applicant: Source Production & Equipment Co., Inc., St. Rose, LA (US)

(72) Inventor: John J. Munro, III, North Andover, MA (US)

(73) Assignee: Source Production & Equipment Co., Inc., St. Rose, LA (US)

(21) Appl. No.: 14/013,300

(22) Filed: Aug. 29, 2013

Related U.S. Application Data
(60) Provisional application No. 61/694,313, filed on Aug. 29, 2012.

(51) Int. Cl. A61N 5/10

(52) U.S. Cl. CPC A61N 5/1077
USPC 600/1; 250/492.1

(57) ABSTRACT

A system provided for delivering Accelerated Partial Breast Irradiation (APBI) and for delivering a boost to standard whole-breast irradiation (WBI) for the treatment of breast cancer that significantly reduces the risks of adverse cosmetic outcomes and toxicities. This is achieved by a method and device for delivering a uniform radiation dose to the target volume with significantly reduced dose to the non-target volume, skin and chest wall of the ipsilateral breast, and virtually no dose to the contralateral breast, lungs, and heart.

### RELATED APPLICATIONS

This application claims priority to U.S. provisional patent application 61/694,313, filed on Aug. 29, 2012, and entitled “RADIATION THERAPY OF PROTRUDING AND/OR CONFORMABLE ORGANS,” which is incorporated by reference herein.

### TECHNICAL FIELD

This application is related to the field of radiation therapy.

### BACKGROUND OF THE INVENTION

Breast cancer is the most common malignancy among women in the United States with an estimated 226,870 new cases in 2012 and about 39,510 deaths from this disease. Standard treatment for early stage cancer is breast conservation, consisting of lumpectomy and six weeks of daily whole-breast irradiation (WBI), which has proven local control and survival rates similar to mastectomy, while providing superior cosmetic outcome and less psychological and emotional trauma. However, potential side effects from radiation dose to organs adjacent to the breast (lungs, heart and scattered radiation dose to the contralateral breast) are a concern. Moreover, a protracted course of WBI presents logistical problems to many elderly patients and patients who live a significant distance from treatment centers. Despite obvious cosmetic and potential psychological and emotional advantages of breast conservation treatment, only ˜40% of patients who are candidates for breast conservation actually receive it.

A newer approach, Accelerated Partial Breast Irradiation (APBI), has been used to deliver a course of radiation therapy in 4-5 days of twice-daily treatments, significantly shortening the overall treatment duration. This decreases the burden of care for breast conservation patients, eliminates many logistical problems (including integration of local and systemic therapies), makes this option available to more women and potentially reduces health care costs. Additionally, toxicity to adjacent normal structures (i.e., heart, underlying chest wall, contralateral breast) should be reduced significantly by decreasing the volume of irradiated tissue. This approach is the subject of ongoing NSABP/RTOG clinical trials and has been deemed suitable by ASTRO for a limited subset of breast cancer patients outside of the clinical trials.

APBI has been tested as the sole method of irradiation following lumpectomy in numerous trials. Five-year results from the majority of these trials have demonstrated local control rates comparable to those observed after conventional WBI. These reports suggest that APBI is comparable to whole-breast irradiation in both safety and efficacy.

APBI has been delivered using three broad techniques, and each has its shortcomings.

The longest experience of APBI is with multicatheter brachytherapy which has achieved excellent local control (from 0.3% to 0.8%) and good cosmetic outcome reported after at least 6-12 years of follow-up. However, the interstitial technique is very practitioner-dependent requiring a great deal of skill to be implemented successfully. It has been found that, in general, the implant volume, the volume of tissue receiving doses of 150% and 200% of the prescription dose (V150 and V200) and the global dose homogeneity (DHI) were strongly correlated with adverse outcome such as increased risk of late skin toxicity, late subcutaneous toxicity and clinically evident fat necrosis.

More recently, intracavitary balloons and cage-like devices have been extensively used to deliver high dose rate (HDR) brachytherapy. One example is the MammoSite™ device with which more than 50,000 patients have been treated, but other devices are also in the marketplace. Intracavitary balloons have been promoted as much easier and technically less demanding than the multicatheter technique. However, with longer follow-up time, some drawbacks and limitations of this technique have emerged, including lack of conformance of the balloon to the cavity and to the asymmetrical target, high rate of balloon explantation, discomfort, wound problems, pain, early skin reactions with moist desquamation, infection, clinically significant and persistent seroma, and high costs, which have served to temper somewhat the enthusiasm of the early experiences. Early local control results are not as favorable as after multicatheter brachytherapy. There is now accumulating evidence showing a progressive decrease in excellent and good cosmetic outcome when follow-up extends beyond five years, related to seroma formation infection rate and skin-balloon distance.

The third APBI technique is three-dimensional conformal radiation therapy (3D-CRT). Typical 3D-CRT includes 3-5 noncoplanar fields with no beams directed towards the heart, lung or contralateral breast. 3D-CRT eliminates the additional surgical procedure and improves dose homogeneity within the target volume, which may improve cosmetic results and reduce the risk of symptomatic fat necrosis, but does so at the expense of irradiating more normal tissue. Unlike brachytherapy, which requires additional training, most radiation facilities already have the technologic tools required to deliver 3D-CRT. The primary disadvantage is that larger volumes of breast need to be included in the target to account for the intrinsic intra- and interfraction motion, uncertainty in target delineation and setup uncertainties in order to avoid improper target coverage. PTV volumes have been reported 5-6 times larger with 3D-CRT than with brachytherapy techniques, with the chest wall/rib receiving 105% of Prescription Dose, the lung receiving 94% and the skin receiving 104%. Recent clinical data has suggested that the 3D-CRT technique is associated with unacceptable toxicities including subcutaneous fibrosis and pneumonitis, and unacceptable cosmesis, all correlating to the volume of normal tissues being excessively irradiated.

Each of the APBI techniques has shortcomings that can lead to adverse cosmetic outcomes, increased risk of skin and subcutaneous toxicities, fat necrosis, or increased risk to other organs due to radiation dose outside the field.

To overcome some of these problems, a technique and device (called AccuBoost) has been developed to peripherally apply breast brachytherapy without piercing the skin as currently performed with interstitial and MammoSite™ applications. Reference is made, for example, to U.S. Pat. No. 8,182,410 B2 to Sioshansi et al., entitled “Peripheral Radiotherapy of Protruding Conformable Organs,” which is incorporated herein by reference. Sioshansi et al. describe that by virtue of being a protruding and deformable organ, the breast lends itself to peripheral brachytherapy by non-invasive applicators. A delivery system exists to implement this developmental treatment modality using real-time mammographic image guidance for stereotactic applicator positioning and CTV localization. In this design, therapeutic dose to the lumpectomy cavity is delivered by externally placing opposing plaque-like applicators at multiple orientations to provide conformity while not exceeding the skin toxicity threshold. The initial assessment of this system determined that dose to lungs, heart, and other critical organs was typically much lower than form 3D-CRT techniques and suggested that this technique may be an attractive APBI option.

A drawback to the AccuBoost approach is the non-uniform dose distribution within the target. In the AccuBoost technique, the dose is delivered to breast tissue that is compressed by a mammography unit. A tungsten shield, in the form of a re-entrant cylinder, is positioned on the compression plate of the mammography unit and a typical ¹⁹²Iridium (Iridum-192 or Ir-192) high dose rate (HDR) brachytherapy source may be manipulated around the inside circumference of this tungsten shield to deliver the dose. A recent applicator design is a reentrant cylinder augmented with an internal truncated cone (frustrum). By placing the truncated cone in the center of the circular applicator, shielding is provided toward much of the skin from each stopping position. This design reduces the skin dose with minimal effect on the dose to the treatment plane.

Although this technique achieves the objective of significantly reducing dose to the non-target volume, skin and chest wall of the ipsilateral breast and virtually no dose to the contralateral breast, lungs, and heart, it delivers a non-uniform dose to the target itself. This non-uniformity can have the result of under-dosing critical target tissue and thereby reducing the therapeutic effect, or, in order to compensate for this reduction, over-dosing other target tissue, and thereby increasing the probability of unacceptable toxicities such as subcutaneous fibrosis and pneumonitis, and unacceptable cosmesis.

Accordingly, it would be desirable to provide a radiotherapy system that will significantly reduce the risks of adverse cosmetic outcomes and toxicities by delivering a uniform radiation dose to the target volume with significantly reduced dose to the non-target volume, skin and chest wall of the ipsilateral breast and virtually no dose to the contralateral breast, lungs, and heart. It would further be desirable to irradiate only the breast with an extremely uniform radiation dose, achieve dose distributions that will significantly reduce the risks of adverse cosmetic outcomes and toxicities, and reduce costs (both initial capital outlay and operational). This would have a significant impact on the treatment of breast cancer.

### SUMMARY OF THE INVENTION

According to the system described herein, a radiotherapy device includes a shield. A single radiation source is disposed within the shield. The single radiation source is movable within a channel of the shield. A collimated opening is disposed in the shield that enables the single radiation source to be moved along the channel and positioned in an exposed position within the shield. A beam modulator component may be disposed adjacent to the collimated opening. The single radiation source may include Se-75 and/or the single radiation source may include Co-56, Co-57, Co-58, Co-60, Zn-65, Pd-103, Cd-109, I-125, Cs-131, Cs-137, Sm-145, Gd-153, Yb-169, W-187, Ir-192, and/or Au-198. The collimated opening may have a conical shape. The shield may be made of a material having a density greater than 6 g/cm³.

According further to the system described herein, a method of performing radiotherapy includes disposing a single radiation source within a shield. The single radiation source is movable within a channel of the shield. The single radiation source is moved along the channel into an exposed position above a collimated opening of the shield. A uniform radiation dose is delivered from the single radiation source to a target volume. The method may further include flattening the radiation beam before delivery to the target volume using a beam modulator component disposed adjacent to the collimated opening of the shield. The single radiation source may include Se-75 and/or the single radiation source may include Co-56, Co-57, Co-58, Co-60, Zn-65, Pd-103, Cd-109, I-125, Cs-131, Cs-137, Sm-145, Gd-153, Yb-169, W-187, Ir-192, and/or Au-198. The collimated opening may have a conical shape. The shield may be made of a material having a density greater than 6 g/cm³. The method may further include imaging the target volume and the imaging of the target volume may be performed using the single radiation source. The single radiation source may be a first single radiation source and delivering the uniform radiation dose may include delivering a radiation dose from a second single radiation source located on an opposite side of a target volume with respect to the first single radiation source.

According further to the system described herein, a radiotherapy system includes a first radiotherapy device and a second radiotherapy device disposed on an opposite side of a target volume with respect to the first radiotherapy device. Each of the first radiotherapy device and the second radiotherapy device includes a shield; a single radiation source disposed within the shield; and a collimated opening disposed in the shield that enables the single radiation source to be positioned in an exposed position within the shield. The first radiotherapy device and/or the second radiotherapy device may include a beam modulator component disposed adjacent to the collimated opening. The single radiation source of the first radiotherapy device and/or the single radiation source of the second radiotherapy device may include Se-75. One of the first radiotherapy device or the second radiotherapy device may act as a beam catcher for the other of the first radiotherapy device or the second radiotherapy device. An imaging system may further be provided that images the target volume using radiation from the single radiation source of the first radiotherapy device and/or the second radiotherapy device.

### BRIEF DESCRIPTION OF THE DRAWINGS

Embodiments of the system described herein will now be explained in more detail in accordance with the figures of the drawings, which are briefly explained as follows.

FIGS. 1A and 1B are schematic illustrations showing a shielded radiotherapy device according to an embodiment of the system described herein in which a single radiation source may be positioned with respect to a collimated conical opening within a shield.

FIG. 2 is a graph of a relative dose equation for a source located above a reference plane.

FIGS. 3A and 3B are schematic illustrations showing a shielded radiotherapy device according to an embodiment of the system having components like that described in connection with FIGS. 1A and 1B and further incorporating a beam modulator element.

FIG. 4 is a graph of lateral dose rate distribution at a central plane according to an embodiment of the system described herein.

FIG. 5 is a graph showing dose rate distributions of an embodiment of the system described herein at other depths.

FIG. 6 is a schematic illustration showing the use of two radiotherapy devices according to an embodiment of the system described herein.

FIG. 7 is a schematic illustration showing the use of a VMAT apparatus in connection with an embodiment of the system described herein.

FIGS. 8A-8C show histograms of dosimetric results of the Monte Carlo simulations in connection with an embodiment of the system described herein.

FIG. 9 is a schematic illustration showing that imaging may also be incorporated within the system according to an embodiment of the system described herein.

### DETAILED DESCRIPTION OF VARIOUS EMBODIMENTS

According to the system described herein, a method is provided for delivering Accelerated Partial Breast Irradiation (APBI) and for delivering a boost to standard whole-breast irradiation (WBI) for the treatment of breast cancer that will significantly reduce the risks of adverse cosmetic outcomes and toxicities. This is achieved by delivering a uniform radiation dose to the target volume with significantly reduced dose to the non-target volume, skin and chest wall of the ipsilateral breast, and virtually no dose to the contralateral breast, lungs, and heart.

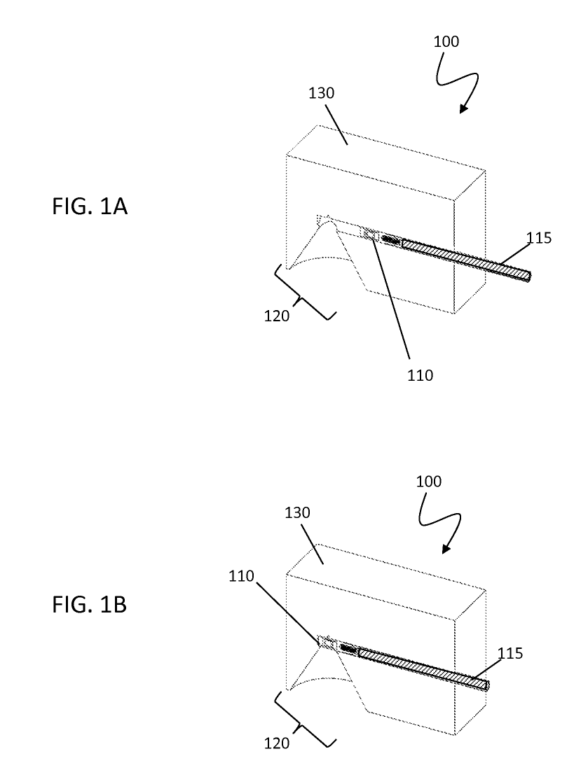

FIGS. 1A and 1B are schematic illustrations showing a shielded radiotherapy device 100 according to an embodiment of the system described herein in which a single radiation source 110 may be positioned with respect to a collimated conical opening 120 within a channel of a shield 130. FIG. 1A shows the radiation source 110 in a shielded position in the radiotherapy device 100. FIG. 1B shows radiation source 110 moved by an arm 115 in the channel of the shield 130 into an exposed position above the collimated conical opening 120 in the radiotherapy device 100. Although the collimated opening 120 is shown as a right-circular cone, other shapes are possible and may be appropriately used in connection with the system described herein. For example, the collimated conical opening 120 may have other conical shapes, such as that of a pyramid and/or other volume shape having a polygonal base. In various embodiments, the shield 130 may be made of tungsten. Other appropriate shielding materials may be used, such as uranium or lead. More generally, other materials having high densities, such as a density greater than 6 g/cm³, may be used for the shield, such as lead, steel, brass, copper, silver, gold and/or tantalum, for example.

In any plane normal to the axis connecting that plane to the source of radiation, the dose distribution will vary as a function of radial distance from the axis due to the inverse square behavior of dose (and dose rate) distribution. For example, the dose at any point in the plane at a distance r from the axis connecting that plane to the source of radiation, relative to the dose at the axis, can be expressed as:

$$Relative Dose = \frac{( {r}^{2} + {d}^{2} )}{{d}^{2}} \qquad EQUATION 1$$

where:
r: radial distance from the axis within the plane, and
d: distance from the source to the plane

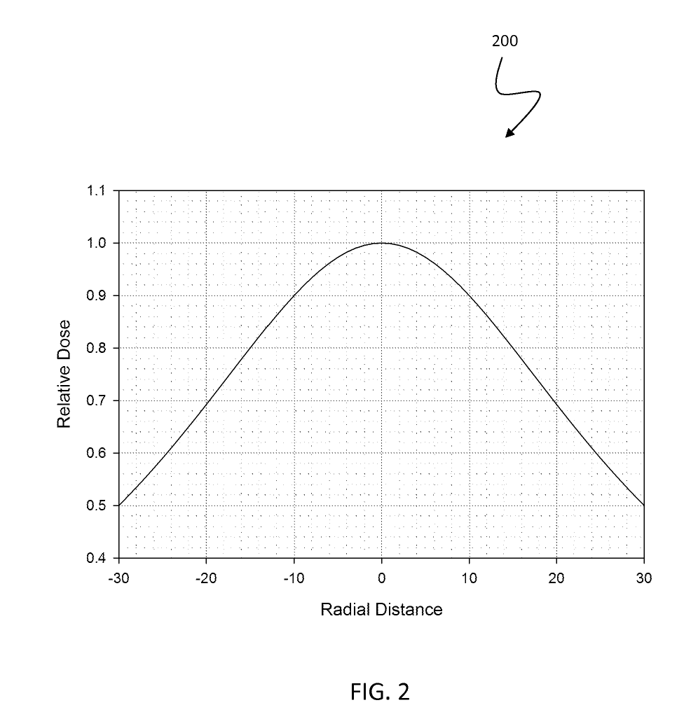

FIG. 2 is a graph 200 of Equation 1 for a source located above a reference plane. In the illustrated embodiment, the source is located 30 mm above the reference plane.

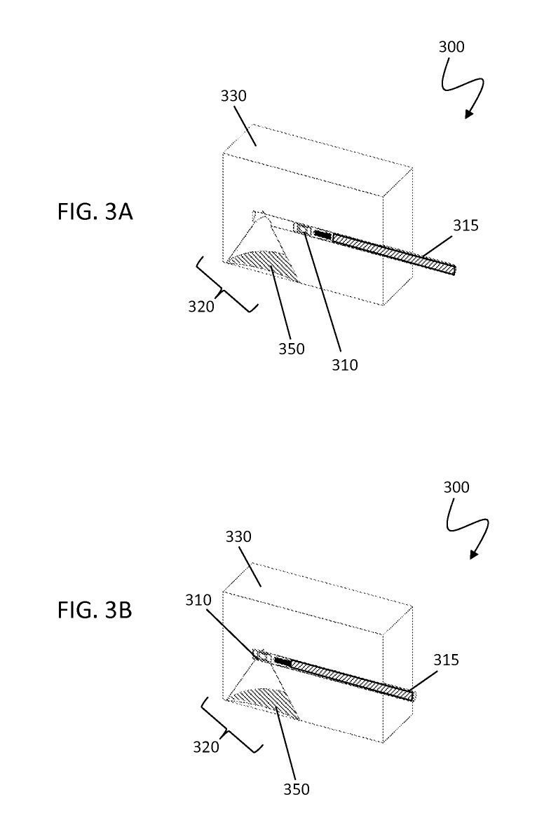

FIGS. 3A and 3B are schematic illustrations showing a shielded radiotherapy device 300 according to an embodiment of the system having components like that described in connection with FIGS. 1A and 1B and further incorporating a beam modulator element 350. In each figure, a single radiation source 310 may be positioned with respect to a collimated conical opening 320 within a channel of a shield 330. FIG. 3A shows the radiation source 310 in a shielded position in the radiotherapy device 300 with the beam modulator element 350 in position adjacent to the opening 320. FIG. 3B shows radiation source 310 moved in the channel of the shield 330 by an arm 315 into an exposed position in the radiotherapy device 300 with the beam flattener element 350 in position adjacent to the opening 320. Although the collimated opening 320 is shown as a right-circular cone, other shapes are possible and may be appropriately used in connection with the system described herein. For example, the collimated conical opening 320 may have other conical shapes, such as that of a pyramid and/or other volume shape having a polygonal base. In various embodiments, the shield 130 may be made of tungsten. Other appropriate shielding materials may be used, such as uranium or lead. More generally, other materials having high densities, such as a density greater than 6 g/cm³, may be used for the shield, such as lead, steel, brass, copper, silver, gold and/or tantalum, for example.

The beam modulator component 350 enables control of an intensity of the beam. In an embodiment, the beam modular component 350 may be a beam flattener that controls the beam intensity to be uniform in all locations and/or directions. In another embodiment, the beam modulator component 350 may enable control of the beam intensity in a non-uniform manner. For example, the beam modulator component 350 may allow a higher radiation intensity in a center of a target volume (tumor) and a lower radiation intensity at the periphery of the target volume. In an embodiment, the beam modulator component 350 may be made of a similar material as that of the shield 320.

A series of dosimetry calculations (Monte Carlo simulations) have been made to compare the design of the system described herein to the AccuBoost conical applicator as described in Yang Y, Rivard M J, “Dosimetric optimization of a conical breast brachytherapy applicator for improved skin dose sparing,” Med Phys. 2010 November; 37(11):5665-71, which is incorporated herein by reference. The chosen parameters are those described by Yang and Rivard as the optimal cone applicator, with an inside diameter of 60 mm and an inside height of 26 mm. In order not to bias the results by differences in the Monte Carlo techniques, the conical applicator was modeled and simulated using the same dosimetry calculation methodology that was used to model and simulate the system described herein. With each of these dose delivery methods, the dose distribution was calculated throughout the breast, with 60 mm separation between the compression plates and over a cylindrical volume with a radius of 60 mm.

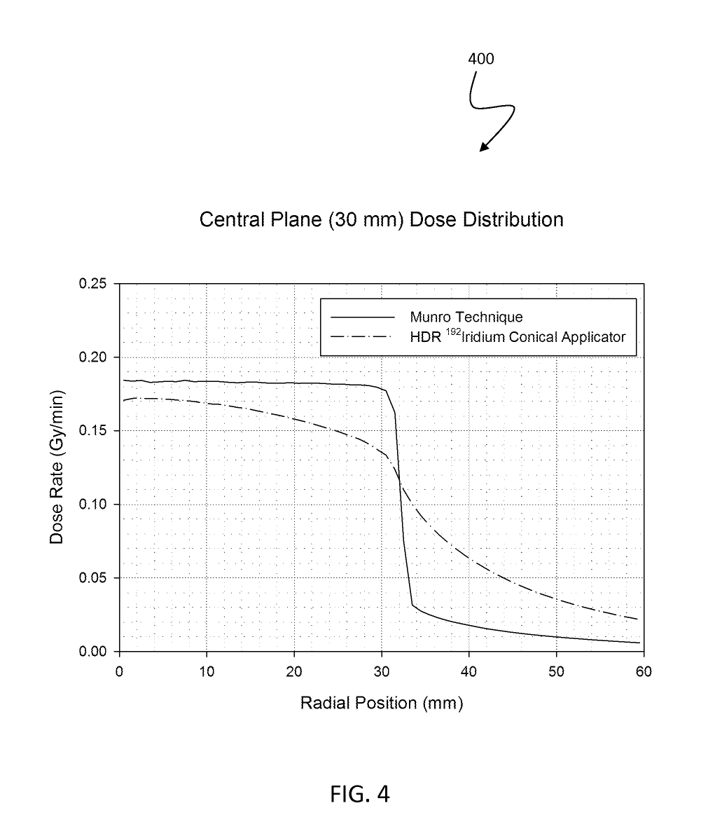

FIG. 4 is a graph 400 of the lateral dose rate distribution at the central plane (at a depth of 30 mm from the surface of the breast) according to an embodiment of the system described herein. The dose rate results are absolute values (Gy/min) and not relative values. The results for the HDR Ir-192source are based on a source of 10 Ci stepping around the entire inner circumference of the conical applicator. The results for the system described herein for the embodiment of the device like that shown in FIGS. 3A and 3B (identified as Munro Technique) are calculated using the maximum proposed activity. These results represent exposure from one side only, and do not include the effects of opposing exposures. As is shown in this graph 400 of FIG. 4, the dose distribution within the target region is significantly flatter, with a sharper demarcation at the edges, as a result of use of the beam modulator component 350. Also, the absolute dose rate is somewhat higher (˜10%).

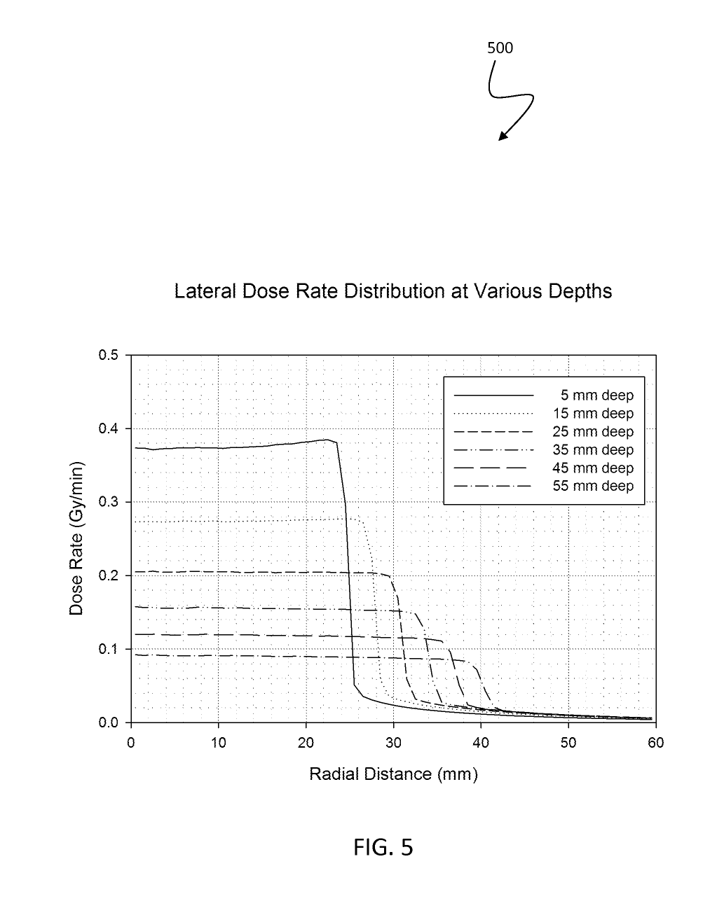

FIG. 5 is a graph 500 showing dose rate distributions of an embodiment of the system described herein at other depths. The results show that the flat dose rate distribution is not an anomaly occurring at the depth of 30 mm, but exists at other depths, for example, 5 mm, 15 mm, 25 mm, 35 mm, 45 mm and 55 mm. It is also noted that this is not restricted to a target volume radius of 30 mm. This flat dose rate distribution may be achieved at virtually all target sizes. It is also not restricted to circular targets. This flat dose rate distribution may be achieved in irregularly-shaped volumes as well.

In various embodiments, it is noted that the flat dose distribution of the system described herein may be achieved with multiple types of radiation sources. The current AccuBoost system employs an Ir-192 HDR brachytherapy source, and such an Ir-192 source may be used in the system described herein. However, it is noted that the beam-modulating is rendered more efficatious with lower-energy radiation sources. As described in U.S. Pat. No. 8,182,410 to Sioshansi et al., cited elsewhere herein, radionuclide(s) of the source(s) may be chosen from the list of commonly recognized and/or available radionuclides. The ideal isotope may have the right combination of half-life, gamma ray energies and ease of production and purification. The half-life has an impact on the shelf life of the product. The x-ray or gamma ray (photon) energies control the depth of the field for dose delivery and may be optimized such that it matches the volume and location of the tumor bed. Higher energy photons are better for more deeply seated targets. The radionuclide may be chosen among available or easily producible species. Example options for radioisotopes capable of meeting these requirements discussed in Sioshansi et al. include Co-56, Co-57, Co-58, Co-60, Zn-65, Pd-103, Cd-109, I-125, Cs-131, Cs-137, Sm-145, Gd-153, Yb-169, W-187, Ir-192, and Au-198.

According to an embodiment of the system described herein, another suitable radio-isotope that may be beneficially used as the radiation source in the system described herein is ⁷⁵Selenium (Selenium-75 or Se-75). Se-75 decays by electron capture accompanied by the emission of gamma rays with energies in the range of 120 keV −400 keV (average energy: 215 keV). Se-75 is an advantageous choice for a gamma radiation source in connection with the system described herein because high specific activities (up to 1500 Ci/g) can be achieved. Also, Se-75's half-life is 120 days requiring less frequent source replacement than Ir-192 (t_{1/2}=74 days). For further discussion of radiation sources, including Se-75, reference is made to U.S. Pat. No. 8,357,316 B2 to Munro, III et al., entitled “Gamma Radiation Source,” and U.S. Pub. No. 2013/0009120 A1 to Munro, III et al., entitled “Radioactive Material Having Altered Isotopic Composition,” which are incorporated herein by reference. Reference is also made to U.S. Pat. No. 6,875,377 B1 to Shilton, entitled “Gamma Radiation Source,” which is incorporated herein by reference.

According to the system described herein, a single stationary Se-75 source located on the central axis, will achieve comparable skin dose and comparable treatment time to the AccuBoost circumferential Ir-192 HDR brachytherapy source technique.

In an embodiment, the Se-75 source may be delivered in a radiotherapy device, like the radiotherapy devices 100 or 300 that are further discussed elsewhere herein, using tungsten for shielding. The package may have a diameter of ˜75 mm (3 inches) and weigh ˜5.4 kg (12 lbs) which would be sufficiently light as to be capable of mounting on the compression plate of a mammography system. If the device were limited to 80 Curies, then it would be transported as a Type A container, minimizing the regulatory burden. Using this approach, it would be possible to use two units, mounted in opposing positions, simultaneously to reduce the treatment time in half.

The use of Se-75 with its lower photon energies also reduces the room shielding requirements over those of an Ir-192 HDR brachytherapy source. Because of the self-contained storage device and collimator, there is no need for the source to traverse unshielded between the storage device and the exposing position, as is the case with the Ir-192 HDR source technique.

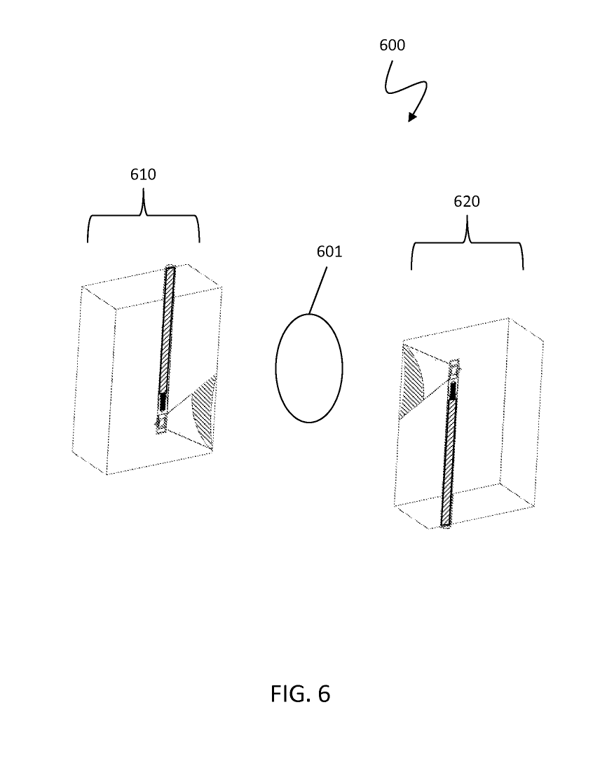

FIG. 6 is a schematic illustration 600 showing the use of two radiotherapy devices 610, 620 according to an embodiment of the system described herein. Unlike the AccuBoost technique in which treatments are typically sequentially made from opposing sides of the compressed breast, the system described herein enables two radiotherapy devices 610, 620, like that of the devices 100 or 300 described elsewhere herein, to be mounted simultaneously on mammography compression plates on both sides of a target volume 601, such as a breast or other organ. This would permit both exposures to be performed simultaneously, significantly reducing the treatment time. Further, the opposing shielded device may act as a beam catcher for the device on the opposite side, providing shielding for the beam emerging from the opposite source and additionally reducing the room shielding requirement.

The foregoing description has been directed to a system for delivering Accelerated Partial Breast Irradiation (APBI) and for delivering a boost to standard whole-breast irradiation (WBI) for the treatment of breast cancer and described in the context of an AccuBoost treatment where the breast is compressed between a pair of compression plates of a mammography system. However, the system described herein is not limited to that configuration.

According to another embodiment, the system described herein may be used in connection with a volumetric modulated arc therapy (VMAT) technique in which only the breast is irradiated to achieve dose distributions that significantly reduces the risks of adverse cosmetic outcomes and toxicities, reduce cost (both initial capital outlay and operational). The VMAT approach places the patient in a prone position and rotationally irradiate only the breast. For a discussion of the VMAT technique, reference is made to Glick S J, “Breast C T,” Annu Rev Biomed Eng. 2007; 9:501-26, which is incorporated herein by reference.

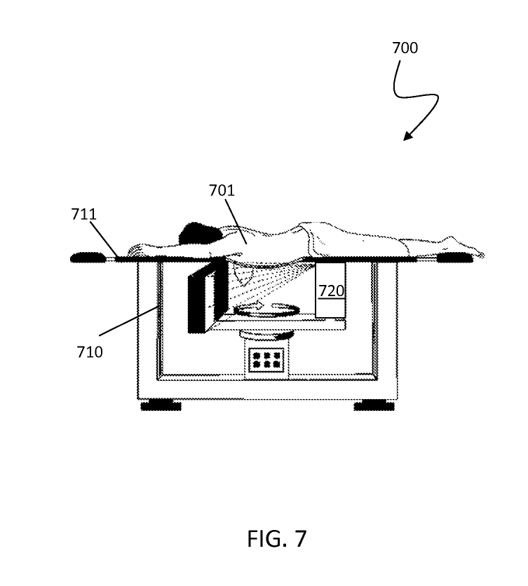

FIG. 7 is a schematic illustration 700 showing the use of a VMAT apparatus 710 in connection with an embodiment of the system described herein. A patient 701 is placed in a prone position on the apparatus 710 that rotationally irradiates only the breast and incorporates simultaneous (or near-simultaneous) CT-imaging of the target in exactly the same position as the treatment delivery. The patient 701 would lie on a shielded table 711 with the breast protruding below the shielded surface to assure that no direct radiation dose would be delivered to the contralateral breast, lung or the heart. A radiotherapy device 720, like that of the radiotherapy devices 100 or 300 discussed elsewhere herein, causes the radiation source to be directed only at the breast such that no primary radiation would be directed at the patient's chest wall, lung or heart. In those cases where dose needs to be delivered close to the chest wall, proper design of the table 711, including a trough, may achieve good coverage of the breast and axilla.

In some cases, external beam APBI may only be performed using high energy photons, principally because of the need to deliver the beam through long path lengths in the body without creating very high skin/entrance doses. Breast radiation therapy is typically performed with high energy radiation accelerators which deliver photons with energies of many thousands of keV (many MeV). However, by irradiating the breast only, through this prone-positioned volumetric modulated arc therapy, the skin dose will be well within the acceptable guidelines while achieving very uniform prescription doses in the target.

Through the use of the system described herein, radiation therapy may be applied with a radiation source that may include any of the radionuclide sources identified above, especially including Se-75. An advantage of the delivery approach according to the system described herein is the ability to use low-energy radiation. Earlier considerations of rotational breast therapy have focused on higher energy X-ray sources (320 kVₚ orthovoltage tubes). However, the combination of rotation and collimation limits the skin dose; only small areas of the skin are in the near field beam for only very short fractions of the treatment duration. This permits the treatment to be performed using relatively low energies; energies that would not generally be considered for volumetric treatment. As described below, acceptable results have been obtained with energies as low as 120 kVₚ. By using variable (multi-leaf) collimators, the radiation beam may be adjusted to conform directly to the target volume at all angular positions.

The use of low-energy radiation sources leads to a second important innovation: the incorporation of simultaneous (or near-simultaneous) CT-imaging of the target in exactly the same position as the treatment delivery. Breast CT has been studied for some time and systems have been built to demonstrate feasibility, but the approach of the system described herein would incorporate the use of CT imaging into the therapy system using the same X-ray source. This would assure precise target location and avoid the difficulties of other external beam techniques in reliably reproducing the target from fraction to fraction.

A treatment facility according to the system described herein may be small and self-contained so that it could be installed in an unshielded treatment room, permitting the therapist to be present in the same room as the patient during treatment. The patient will be able to view her surroundings, avoiding the anxiety resulting from the feeling of being closed in that is so common in MRI and CT examinations and external beam therapy. It will provide the additional benefit of allowing clinical personnel to approach the patient for comfort and care during the procedure, which is now not possible without interruption/termination of the treatment. Most importantly, this treatment facility would be less costly that alternative external beam machines, allowing this procedure to be more widely available.

Monte Carlo techniques (MCNP5) were used to simulate the dose distribution in a breast under several treatment scenarios. The treatment geometry is similar to that shown in FIG. 7. For simplicity, the breast was postulated to be a hemispherical section with a diameter of 140 mm superimposed onto a cylindrical section with a diameter of 140 mm and a length of 70 mm. A 20 mm radius spherical lumpectomy cavity was located concentric with the hemisphere. The target volume was postulated as a 10 mm thick spherical shell surrounding the lumpectomy cavity. Irradiations were simulated with 120 kVₚ and 160 kVₚ X-ray sources, each located at 500 mm from the center of the lumpectomy cavity.

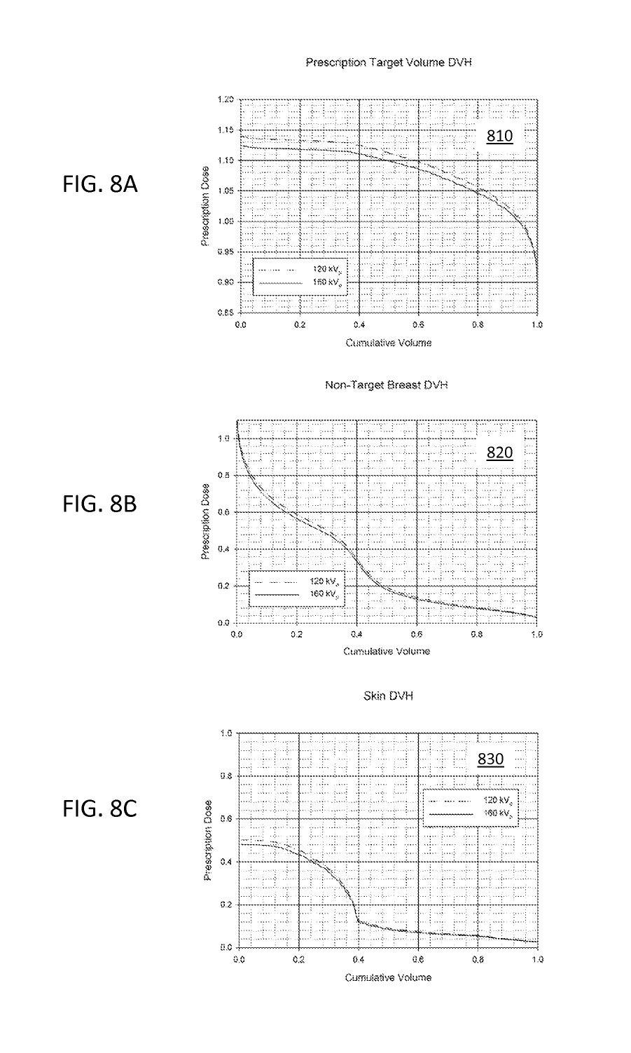

FIGS. 8A-8C show histograms of dosimetric results of the above-noted simulations. FIG. 8A shows a target dose-volume histogram (DVH) 801. FIG. 8B shows a non-target breast DVH 802. FIG. 8C shows a skin DVH 803. To assess the significance, these results were compared to the dosimetric guidelines for 3D-CRT used in the NSABP B-39 protocol for APBI. However, as noted above, recent clinical data has suggested that the current 3D-CRT technique is associated with unacceptable toxicities and unacceptable cosmesis, correlating to the volume of normal tissues being excessively irradiated. Accordingly, the results are also compared with more stringent dose-volume constraints for toxicity avoidance: 120 kVₚ and 160 kVₚ. The comparison results are presented in Table 1:

TABLE 1
Summary of Dosimetry Parameters

| | NASBP B-39 | 120 kVₚ | 160 kVₚ |
|---|---|---|---|
| Target V90 | ≧90% | 100.0% | 100.0% |
| Target Maximum | <120% | 114.0% | 112.5% |
| Ipsilateral Breast V100 | <35% | 5.0% | 4.9% |
| Ipsilateral Breast V50 | <60% | 33.6% | 31.2% |
| Contralateral Breast | <3% | 0.0% | 0.0% |
| Ipsilateral Lung V30 | <15% | | |
| Contralateral Lung V5 | <15% | 0.0% | 0.0% |
| Skin (Maximum Dose) | N.S. | 50.3% | 48.2% |
| Chest Wall/Rib (Max) | N.S. | 28.2% | 26.5% |

The system described herein beneficially achieves dosimetric results that could significantly reduce the risks of adverse cosmetic outcomes and toxicities in APBI and also reduce risk in boost of WBI.

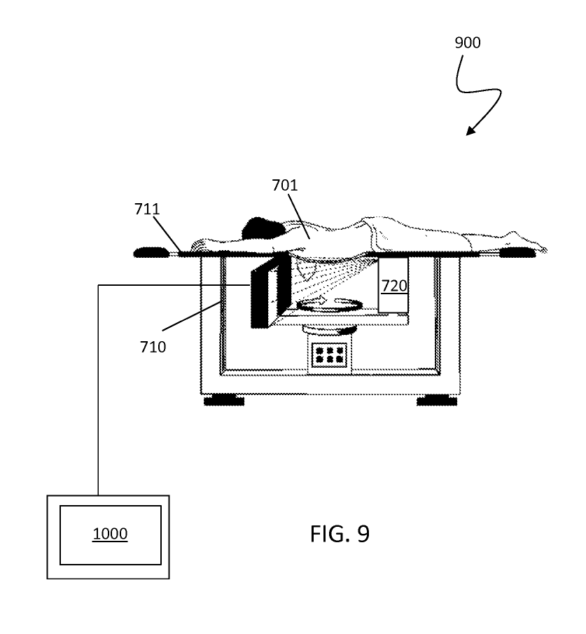

FIG. 9 is a schematic illustration 900 showing that imaging may also be incorporated within the system according to an embodiment of the system described herein. The illustration 900 shows VMAT components like that of the illustration 700 described in connection with FIG. 7 and further shows an imaging system 1000. The delivery approach lends itself to simultaneous (or near-simultaneous) imaging of the target, using the imaging system 1000, in exactly the same position as treatment delivery. With this addition, breast CT may be performed immediately before the therapy, thereby assuring target location and avoiding the difficulties of reliably reproducing the target from fraction to fraction in other external beam techniques. In an embodiment, the same radiation source maybe used for imaging and therapy. The imaging may include the addition of an imaging plate to the apparatus 610 to perform cone-beam CT. Alternatively, an additional radiation source can be incorporated for imaging, likely orthogonally to the therapy beam in order to make sequential cone-beam CT images to be very immediately followed by VMAT. The imaging system may further be used in connection with the use of multiple radiotherapy devices like that shown in FIG. 6 and in which, in an embodiment, the imaging system may image the target volume using radiation from the single radiation source of the first radiotherapy device and/or the second radiotherapy device.

The foregoing descriptions have been directed to a system for delivering Accelerated Partial Breast Irradiation (APBI), for delivering a boost to standard whole-breast irradiation (WBI) for the treatment of breast cancer and/or for delivering radiation therapy using a VMAT technique. However, the system described herein may be used with other appropriate treatment regimes. Further, the system described herein may be applied to body parts and organs other than breasts, specifically where it is desirable to deliver a uniform radiation dose to the target volume with significantly reduced dose to the non-target volume and surrounding tissue and organs.

Various embodiments discussed herein may be combined with each other in appropriate combinations in connection with the system described herein. Additionally, in some instances, the order of steps in the flowcharts, flow diagrams and/or described flow processing may be modified, where appropriate. Further, various aspects of the system described herein may be implemented using software, hardware, a combination of software and hardware and/or other computer-implemented modules or devices having the described features and performing the described functions. The system may further include a display and/or other computer components for providing a suitable interface with other computers and/or with a user. Software implementations of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media such as a CD-ROM, a DVD-ROM, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A radiotherapy device, comprising:
a shield;
a single radiation source disposed within the shield, wherein the single radiation source is movable within a channel of the shield; and
a collimated opening disposed in the shield that enables the single radiation source to be moved along the channel into an exposed position within the shield.

2. The radiotherapy device according to claim 1, further comprising:
a beam modulator component disposed adjacent to the collimated opening.

3. The radiotherapy device according to claim 1, wherein the single radiation source includes Se-75.

4. The radiotherapy device according to claim 1, wherein the single radiation sources includes at least one of: Co-56, Co-57, Co-58, Co-60, Zn-65, Pd-103, Cd-109, I-125, Cs-131, Cs-137, Sm-145, Gd-153, Yb-169, W-187, Ir-192, and Au-198.

5. The radiotherapy device according to claim 1, wherein the collimated opening has a conical shape.

6. The radiotherapy device according to claim 1, wherein the shield is made of a material having a density greater than 6 g/cm³.

7. A method of performing radiotherapy, comprising:
disposing a single radiation source within a shield, wherein the single radiation source is movable within a channel of the shield;
moving the single radiation source along the channel into an exposed position above a collimated opening of the shield; and
delivering a uniform radiation dose from the single radiation source to a target volume.

8. The method according to claim 7, further comprising:
modulating the radiation beam before delivery to the target volume using a beam modulator component disposed adjacent to the collimated opening of the shield.

9. The method according to claim 7, wherein the single radiation source includes Se-75.

10. The method according to claim 7, wherein the single radiation sources includes at least one of: Co-56, Co-57, Co-58, Co-60, Zn-65, Pd-103, Cd-109, I-125, Cs-131, Cs-137, Sm-145, Gd-153, Yb-169, W-187, Ir-192, and Au-198.

11. The method according to claim 7, wherein the collimated opening has a conical shape.

12. The method according to claim 7, wherein the shield is made of a material having a density greater than 6 g/cm³.

13. The method according to claim 7, further comprising:
imaging the target volume.

14. The method according to claim 13, wherein the imaging of the target volume is performed using the single radiation source.

15. The method according to claim 7, wherein the single radiation source is a first single radiation source and wherein delivering the uniform radiation dose includes delivering a radiation dose from a second single radiation source located on an opposite side of a target volume with respect to the first single radiation source.

16. A radiotherapy system, comprising:
a first radiotherapy device; and
a second radiotherapy device disposed on an opposite side of a target volume with respect to the first radiotherapy device, wherein each of the first radiotherapy device and the second radiotherapy device include:
a shield;
a single radiation source disposed within the shield; and
a collimated opening disposed in the shield that enables the single radiation source to be positioned in an exposed position within the shield.

17. The radiotherapy system according to claim 16, wherein at least one of: the first radiotherapy device or the second radiotherapy device includes a beam modulator component disposed adjacent to the collimated opening.

18. The radiotherapy system according to claim 16, wherein at least one of: the single radiation source of the first radiotherapy device or the single radiation source of the second radiotherapy device includes Se-75.

19. The radiotherapy system according to claim 16, wherein one of: the first radiotherapy device or the second radiotherapy device acts as a beam catcher for the other of: the first radiotherapy device or the second radiotherapy device.

20. The radiotherapy system according to claim 16, further comprising:
an imaging system that images the target volume using radiation from the single radiation source of at least one of: the first radiotherapy device or the second radiotherapy device.
